(19) United States
(12) Plant Patent Application Publication
(10) Pub. No.: US 2007/0016991 P1
(43) Pub. Date: Jan. 18, 2007

(54) AZALEA PLANT NAMED 'MNIJ92'

(76) Inventor: James O. Harris, Lawrenceville, GA (US)

Correspondence Address: JONDLE & ASSOCIATES P.C., 858 HAPPY CANYON ROAD SUITE 230, CASTLE ROCK, CO, 80108, US

(21) Appl. No.: 11/180,478

(22) Filed: Jul. 13, 2005

(51) Int. Cl. A01H 5/00

(52) U.S. Cl. PLT/240

(57) ABSTRACT

A new variety of Azalea plant found as a seedling in a planned cross between the female hybrid Azalea ‘EMBERS’ and the male hybrid Azalea ‘September Morn’ is disclosed. The new variety possesses a unique blooming time and is very cold hardy. It is small, compact, dense and low-growing Azalea with attractive, single red flowers.

### GENUS/SPECIES

Rhododendron sp.

### BACKGROUND OF THE INVENTION

The present invention is a new and distinct variety of evergreen Azalea of the genus Rhododendron. This new Azalea, hereinafter referred to as ‘MNIJ92’, was discovered in March, 1999 in Lawrenceville, Ga. ‘MNIJ92’ originated from a planned hybridization between hybrid Azalea ‘EMBERS’ (U.S. Plant Pat. No. 10,581) and ‘September Morn’ (unpatented) in Lawrenceville, Ga. The value of this new cultivar lies in its unique blooming period, growth habit, and cold hardiness.

Asexual propagation of the new plant by cuttings was performed at the Center for Applied Nursery Research in Dearing, Ga. The new plant retains its distinctive characteristics and reproduces true to type in successive generations by vegetative propagation.

### SUMMARY OF THE INVENTION

The following are the most outstanding and distinguishing characteristics of this new cultivar when grown under normal horticultural practices in Dearing, Ga.
1. Unique spring, summer and fall blooming;
2. Extremely long and floriferous bloom period (5-6 months with blooms);
3. Easily propagated by semi-hardwood cuttings in late spring through summer;
4. Slow growth rate under normal fertilization and moisture conditions;
5. Compact, dense, and low-growing nature;
6. Grows well in containers;
7. Thrives in shade or sun; and
8. Hardy in USDA Zone 7a.

### DESCRIPTION OF THE PHOTOGRAPHS

This new Azalea hybrid variety is illustrated by the accompanying photographs which show blooms and foliage of the plant in full bloom. The colors shown are as true as can be reasonably obtained by conventional photographic means.

FIG. 1. shows growth habit and flowers of mature plant.

FIG. 2. shows a close-up view of stems.

FIG. 3. shows a close-up view of mature foliage, flower buds and flowers.

FIG. 4. shows the plant in full bloom.

FIG. 5. shows a close-up view of several mature inflorescences.

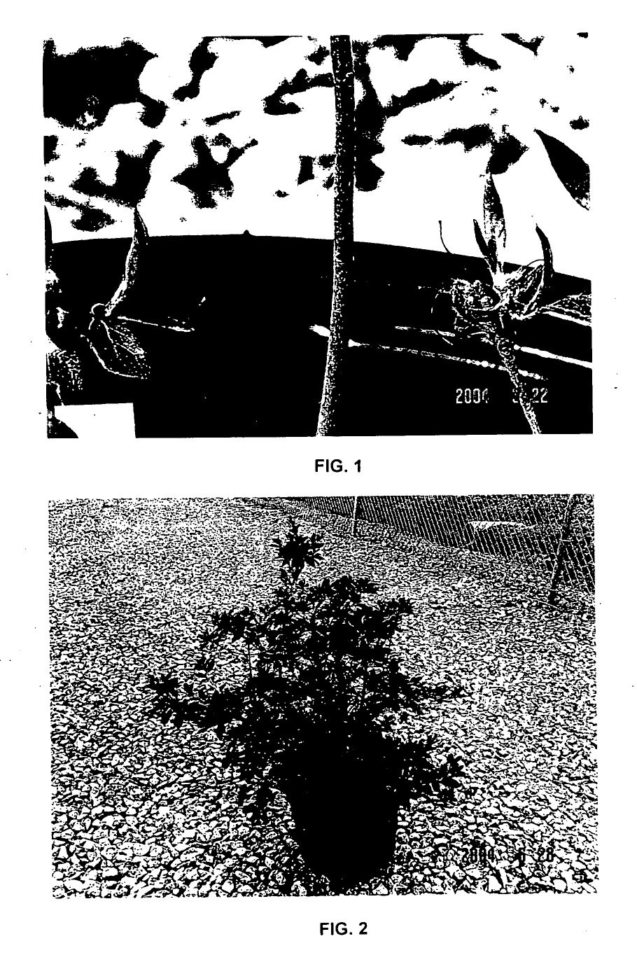

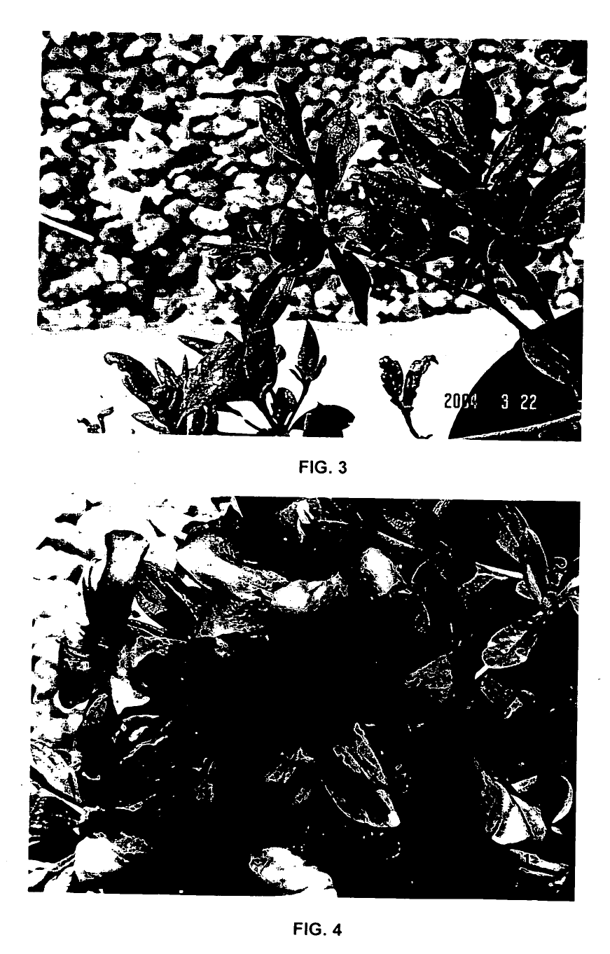

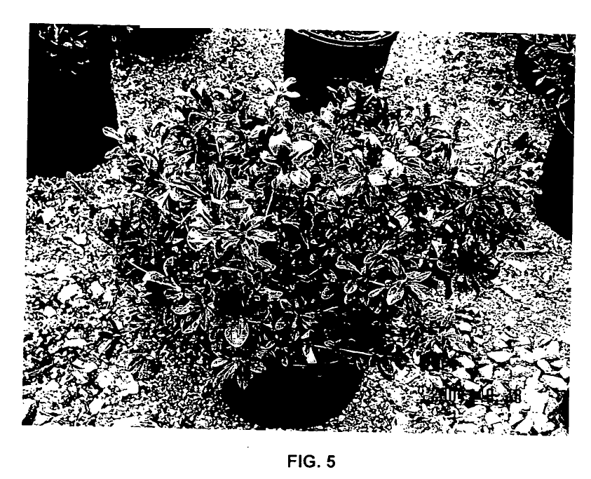

### DETAILED DESCRIPTION OF THE NEW PLANT

The following is a detailed description of the new variety of Azalea based on observations made of a 3-year-old plant grown in a seven-gallon container according to wholesale commercial production conditions in Dearing, Ga. The colors of the various plant parts are detailed with reference to The Royal Horticultural Society Colour Chart.
Classification:
   Family.—Ericaceae.
   Species.—Rhododendron sp.
   Common name.—Azalea.
   Commercial name.—‘MNIJ92’.
Parentage:
   Female parent.—Azalea hybrid ‘EMBERS’ (U.S. Plant Pat. No. 10,581).
   Male parent.—Harris hybrid Azalea ‘September Morn’ (unpatented).
Growth:
   Form.—Small, compact, dense and low growing.
   Height.—12 inches.
   Width.—24 inches.
   Growth habit.—Compact, dense and low growing.
   Growth rate.—Slow growth rate under normal fertilization and moisture conditions. In a period of four years from a rooted cutting, the plant reaches a height of 8 inches and a spread of 20 inches. The growth rate is normally about 2 to 3 inches per year; the plant reaches a height of 12 inches at maturity while maintaining a dense habit due to the abundant branch development.
   Spring growth.—In 2004, the date of initial spring growth was March 9, in Dearing, Ga. After the initial spring flush, there was continuous growth through the fall. The average length of terminal growth of the initial spring flush is about 1½ inches for a plant in full sun and about 1⅝ inches when grown in shade. This growth, if not pruned, will begin to produce flowers starting in mid to late March. As the plant continues to grow through summer and fall, more flower buds are produced which mature and bloom from June until frost. ‘MNIJ92’ bloomed until frost in December of 2003 in Dearing, Ga. The remaining growth produces about 1 to 2 inches of height.
   Life cycle.—Perennial, evergreen.
Leaves: Leaves are evergreen
   Leaf arrangement.—Alternate, simple.
   Leaf shape.—Broadly elliptic to obovate.
   Leaf length.—1⅛ inches.
   Leaf width.— 9/16 inches.
   Leaf margin.—Entire.
   Leaf base.—Acuminate.
   Leaf apex.—Mucronate.
   Petiole length.— 5/16 inch.
   Petiole color.—RHS 146D (yellow-green).
   Mature leaf.—Upper leaf surface: Surface texture: Semi-glossy Surface color: RHS 147A (yellow-green) Surface pubescence: Slight Surface pubescence color: RHS 155C (white) Midveins and laterals: Impressed on surface Lower leaf surface: Surface texture: Matte Surface color: RHS 146B (yellow-green) Surface pubescence: Moderate Surface pubescence color: RHS 164B (greyed-orange) Midveins and laterals: Prominent on surface.
   Immature leaf.—Upper leaf surface: Surface texture: Matte Surface color: RHS 144A (yellow-green) Surface pubescence: Strigose Surface pubescence color: RHS 155C (white) Lower leaf surface: Surface texture: Matte Surface color: RHS 146B (yellow-green) Surface pubescence: Strigose Surface pubescence color: RHS 155C (white) Immature petioles and midveins: Petiole color: RHS 144A (yellow-green) Midvein color: RHS 144A (yellow-green) Petiole pubescence: Strigose Petiole pubescence color: RHS 155C (white) New growth pubescence: Strigose Length of pubescence: 1/32 to 1/16 inch; numerous on mid-vein and petiole, more so as the leaf matures Color of new growth pubescence: RHS 155C (white), maturing to RHS 164B (greyed-orange) throughout the growing season.
Stems: Densely branched
   Young stems.—Stem color: RHS 144B (yellow-green) Stem pubescence: Strigose Stem pubescence color: At point of attachment hairs emerge RHS 155C (white) and mature to RHS 165B (greyed-orange); hairs distal from stem are RHS166D (greyed-orange) Bark color of mature first-year stem: RHS N199C (greyed-brown) Stem length: 1½ to 3 inches Stem diameter: 3/32 to ⅛ inch.
   Second-year stems.—Bark color: RHS 200A (brown) Stem pubescence: Glabrous and rugose Stem length: 1½ to 2½ inches Stem diameter: ⅛ inch.
   Pith.—Solid and uniform.
   Internode length.—Grown in full sun: 1/16 inch to ¾ inch Grown in light shade: 1/16 inch to ⅝ inch.
Flower buds:
   Buds.—Tight; borne in groups of two or three; sheathed by a pair of modified leaf bracts.
   Bud shape.—Ovate and acuminate.
   Bud size.—½×¼ inch.
   Bud scales.—distal portion is pubescent, individual hairs emerge RHS 155C (white) and mature to RHS 167C (greyed-orange).
   Bud color.—RHS 144B and RHS 145D (yellow-green).
   Bud sheath.—Bud sheath size: 7/16 to 17/32 inch long Immature sheath color: RHS 144A (yellow-green) Mature sheath color: As the buds swell, the bud sheath matures from RHS 144A and RHS 145D (yellow-green) to RHS 180B (greyed-red), falls off, and reveals the flower color of RHS 53B (red).
   Bud pedicel.—Pedicel length: 3/16 inch long Pedicel color: RHS 144A (yellow-green) Pedicel pubescence: Strigose Pedicel pubescence color: RHS N155D (white).
   Calyx.—Calyx shape: Cup-shaped, persistent Size: ¼ to 7/16 inch long Color: RHS 144A (yellow-green) Pubescence: Strigose Calyx pubescence color: RHS N155D (white).
Flowers:
   Flower type.—Single, perfect.
   Flowering habit.—Borne on the current season's growth.
   Lastingness of flowers on the plant.—6-8 days in the garden.
   Length of flowering period.—3 to 4 weeks in March and April; flowering resumes in June and continues until frost, which can be as late as November or December in Dearing, Ga.
   Peduncle length.—⅛ to ½ inch.
   Shape.—Open funnel-shaped.
   Flower diameter.—2 to 2½ inches.
   Flower depth.—1½ inches.
   Petal number.—5, fused at the base and remain fused up to 1 inch from the base.
   Petal pubescence.—Glabrous.
   Petal shape.—Elliptic to obovate with rounded apices and entire margins.
   Petal size.—1½ inches long×1 inch wide.
   Petal color.—Upper and lower surfaces: RHS 53C (red) Spots on 3 of the five petals: RHS 53A (red).
   Sepals.—Ovate and joined at the base to form a cup.
   Sepal number.—5.
   Sepal color.—RHS 144A (yellow-green).
   Sepal size.—¼ inch long×⅛ inch wide.
Reproductive parts:
   Ovary.—Extremely tomentose.
   Placenta arrangement.—5 locules.
   Pistil.—Single, non-petaloid.
   Pistil length.—1¼ to 1½ inches long.
   Stigma color.—RHS 58A (red-purple).
   Style color.—RHS 58B (red-purple).
   Stamen number.—5, non-petaloid.
   Stamen length.—1 5/16 to 1 9/16 inches.
   Stamen color.—Filaments: RHS 57A (red-purple) Anthers: RHS 176A (greyed-orange).
   Pollen color.—RHS 155B (white).
   Fragrance.—None.

### Culture

‘MNIJ92’ grows well in a wide range of conditions and tolerates sun to shade. It prefers moist, well-drained soil that is rich in organic matter and responds well to mulching and medium applications of fertilizer. It does best in soil with a pH of 5.0 to 5.5. ‘MNIJ92” is adaptable to container and above ground planters; it also makes a good foundation plant and requires very little pruning. It is propagated with semi-hardwood cuttings in late spring through summer.

### Diseases/Insects

Lace bugs and spider mites can be a problem.

### Comparison with Parental Cultivars

The female, or seed parent, of ‘MNIJ92’ is the hybrid Azalea ‘EMBERS’ (U.S. Plant Pat. No. 10,581) which has strong red, single/semi-double flowers, blooms both early and late seasons, and is a globose-shaped plant. ‘EMBERS’ is the result of a cross between the moderately pink Robin Hill hybrid Azalea ‘Watchet’ (unpatented) and the orange-red Rhododendron oldhamii ‘Fourth of July’ (unpatented).

The male, or pollen, parent is Harris hybrid ‘September Morn’ (unpatented) which is a dense and upright growing, early and late blooming azalea with single, red blooms. ‘September Morn’ is the result of a cross between the red blooming Gartrell hybrid ‘Cherie’ (unpatented) and the yellowish pink blooming Gable hybrid ‘Indian Summer’ (unpatented).

In Table 1 below, the instant plant is shown in comparison with the parental cultivars.

TABLE 1

| | | | ‘September |
|---|---|---|---|
| Characteristic | ‘MNIJ92’ | ‘EMBERS’ | Morn’ |
| Height (Mature) | 12 in. | 4-5 ft. | 2½ ft. |
| Width (Mature) | 24 in. | 5-6 ft. | 3 ft. |
| Flower Diameter | 2-2½ in. | 2½-2¾ in. | 2-2½ in. |
| Flower Form | Single | Single/semi- | Single |
| | | double | |
| Flower Color | RHS 34C (orange- | RHS 44A (red) | RHS 48A (red) |
| | red) | | |
| Flowers per | 3 | 2-3 | 2 |
| Terminal | | | |
| Bloom Period | Mid-March to early | April; Late July | Early April; |
| | April; Mid-June to | to frost | September to |
| | frost | | frost |
| Petal Number | 5 | 5-14 | 5 |
| Hardy Zone | 7 | 7 | 5 |
| Stamen Number | 5 | 0-9 | 5 |
| Stamen Type | Non-petaloid | Some petaloid | Non-petaloid |

What is claimed is:

1. A new variety of Azalea plant named ‘MNIJ92’ as herein shown and described.
